SEQUENCE LISTING (13 sequences)

SEQ ID NO: 1 - protein, 309 aa, Plasmodium falciparum
MVSFSKNKVLSAAVFASVLLLDNNNSAFNNNLCSKNAKGLNLNKRLLHETQAHVDDAHHAHHVADAHHAHHAADAHHAHHAADAHHAHHAADAHHAHHAADAHHAHHAAYAHHAHHAADAHHAHHASDAHHAADAHHAAYAHHAHHAADAHHAHHASDAHHAADAHHAAYAHHAHHAADAHHAADAHHATDAHHAHHAADARHATDAHHAADAHHATDAHHAADAHHAADAHHATDAHHAADAHHATDAHHAADAHHAADAHHATDAHHAHHAADAHHAAAHHATDAHHATDAHHAAAHHEAATHCLRH

SEQ ID NO: 2 - protein, 525 aa, Mus musculus
MKVLTTALLLVTLQCSHALSPTNCDASEPLAEKVLDLINKGRRSGYVFELLRVSDAHLDRAGTATVYYLALDVIESDCWVLSTKAQDDCLPSRWQSEIVIGQCKVIATRYSNESQDLSVNGYNCTTSSVSSALRNTKDSPVLLDFFEDSELYRKQARKALDKYKTDNGDFASFRVERAERVIRARGGERTNYYVEFSMRNCSTQHFPRSPLVFGFCRALLSYSIETSDLETPDSIDINCEVFNIEDHKDTSDMKPHWGHERPLCDKHLCKLSGSRDHHHTHKTDKLGCPPPPEGKDNSDRPRLQEGALPQLPPGYPPHSGANRTHRPSYNHSCNEHPCHGHRPHGHHPHSHHPPGHHSHGHHPHGHHPHSHHSHGHHPPGHHPHGHHPHGHHPHGHHPHGHHPHGHDFLDYGPCDPPSNSQELKGQYHRGYGPPHGHSRKRGPGKGLFPFHHQQIGYVYRLPPLNIGEVLTLPEANFPSFSLPNCNRSLQPEIQPFPQTASRSCPGKFESEFPQISKFFGYTPPK

SEQ ID NO: 3 - protein, 99 aa, Alnus glutinosa
MGYSKTFLLLGLAFAVVLLISSDVSASELAVAAQTKENMQTDGVEEDKYHGHRHVHGHGHGHVHGNGNEHGHGHHHGRGHPGHGAAADETETETETNQN

SEQ ID NO: 4 - protein, 699 aa, Homo sapiens
MGHHRPWLHASVLWAGVASLLLPPAMTQQLRGDGLGFRNRNNSTGVAGLSEEASAELRHHLHSPRDHPDENKDVSTENGHHFWSHPDREKEDEDVSKEYGHLLPGHRSQDHKVGDEGVSGEEVFAEHGGQARGHRGHGSEDTEDSAEHRHHLPSHRSHSHQDEDEDEVVSSEHHHHILRHGHRGHDGEDDEGEEEEEEEEEEEEASTEYGHQAHRHRGHGSEEDEDVSDGHHHHGPSHRHQGHEEDDDDDDDDDDDDDDDDVSIEYRHQAHRHQGHGIEEDEDVSDGHHHRDPSHRHRSHEEDDNDDDDVSTEYGHQAHRHQDHRKEEVEAVSGEHHHHVPDHRHQGHRDEEEDEDVSTERWHQGPQHVHHGLVDEEEEEEEITVQFGHYVASHQPRGHKSDEEDFQDEYKTEVPHHHHHRVPREEDEEVSAELGHQAPSHRQSHQDEETGHGQRGSIKEMSHHPPGHTVVKDRSHLRKDDSEEEKEKEEDPGSHEEDDESSEQGEKGTHHGSRDQEDEEDEEEGHGLSLNQEEEEEEDKEEEEEEEDEERREERAEVGAPLSPDHSEEEEEEEEGLEEDEPRFTIIPNPLDRREEAGGASSEEESGEDTGPQDAQEYGNYQPGSLCGYCSFCNRCTECESCHCDEENMGEHCDQCQHCQFCYLCPLVCETVCAPGSYVDYFSSSLYQALADMLETPEP

SEQ ID NO: 5 - DNA, 36 bp, Artificial Sequence
  Description of Artificial Sequence Synthetic primer
ggtattgagggtcgcatgggcttccaggggccatgg

SEQ ID NO: 6 - DNA, 37 bp, Artificial Sequence
  Description of Artificial Sequence Synthetic primer
agaggagagttagagcctcacgacctgttctgttctc

SEQ ID NO: 7 - DNA, 2127 bp, Mus musculus
ccacgcgtccgccaagacctgaggaagatagagaggcagagagtgggagctataccacgacaaaagggacaatctgaaagtcaaagccaaaaaggcacaaggacccatcagaggcagctgaagccagcctggtcagacgctcagctgctaaacgtccccatgggcttccaggggccatggttgcacacttgtctcctttgggccacagtggccatcctgctggtccctccagtggtgacccaggagttgagaggggccggtctgggcctgggcaactggaacaacaatgcaggcatccctgggtcctcagaggacctatcaactgagtttggtcaccacatccaccggggatatcaaggtgagaaggacagaggccacagagaagagggtgaagacttctccagggaatatggccacagggtccaagaccacaggtaccctggccgcgaggttggagaggagaatgtctctgaagaggtcttcagagggcatgttagacagctccacgggcaccgggaacatgacaatgaagatttaggagactcggcagagaaccacctccccagacagaggagccacagccacgaagatgaggatggcattgtctccagtgagtatcaccgtcacgtccccaggcatgcccaccatggccacggagaggaagatgatgacgatgatggaggagaggaggaggagagggtggatgtgatggaggactctgatgataatgaacaccaggtccatggtcaccagagccactcaaaggagagagatgaactccatcatgcccacagccacaggcaccaaggccacagtgatgatgacgatgacgatggtgtctctactgagcatggacaccaagctcacagatatcaggatcatgaggaggaagacgatggggactcagatgaagacagtcacacccacagagttcaaggccgagaagatgaaaatgatgatgaagacggtgactctggtgaatacagacaccatacccaggaccaccaaggccacaacgaagagcaagatgacgatgatgatgatgatgatgatgatgaagataaagaagactccactgagcaccggcaccagacccaaggccacaggaaggaagaagatgaggatgagtcagatgaagatgatcatcatgtctccaggcatggacgccaaggctatgaagaagaagaagatgatgatgatgatgatggagatgatgactctactgagcatgtgcatcaagcccacagacacagagaccatgagcacaaagatgatgaggatgactcagaagaagactaccatcatgtccccggagtcctccggattgctctctcgactgccagtggggcagccgctgcctactcagcgccttgcctcaacttccccatcagtaccaacaccccctttaccctcgtgtggagcctaagagaacagaacaggtcgtgaagccagcaaagaaaagttctgtcgcgtttgtgaacctttttttttttttaatcaaatcgacaacaaacattaaaactttttttttttaaaaaggacgttaaaaaatttaaaaagtatatgagcttcatgggactaactcatcgccttcccttgcgtacttcagattgtagccatacttttaaaaaaaaaggcaaagaggataatgacattttttatcagtattgtgaataaacttgaacacaaatacagaagttctatgtcctgtcttcagttgtagaagttgtcttctgcaaggtacaaccacccacttgaacttcctctgatgacacaatccacaattctataagggaatcagtgttcacgtctctgtatatatttatttatgtgtaatttaatgggatttgtaaatatggtgagtctgttttaaacctttttttatttatctggtgatctcgtttacctcctgtttagtgggctttggatcctccctgttagttcttcatgtggttttacttagaaatccaaggtttgggtaagactccccctccccaccccttttctccaattcatggatttagccccgtggtagcatgttaaacgattataatgaaacagctgaacaaaaacatttttaaggtaaaataaaaatttatatataattagtaaaaaaaaaaaaaaaaa

SEQ ID NO: 8 - protein, 428 aa, Mus musculus
MGFQGPWLHTCLLWATVAILLVPPVVTQELRGAGLGLGNWNNNAGIPGSSEDLSTEFGHHIHRGYQGEKDRGHREEGEDFSREYGHRVQDHRYPGREVGEENVSEEVFRGHVRQLHGHREHDNEDLGDSAENHLPRQRSHSHEDEDGIVSSEYHRHVPRHAHHGHGEEDDDDDGGEEEERVDVMEDSDDNEHQVHGHQSHSKERDELHHAHSHRHQGHSDDDDDDGVSTEHGHQAHRYQDHEEEDDGDSDEDSHTHRVQGREDENDDEDGDSGEYRHHTQDHQGHNEEQDDDDDDDDDDEDKEDSTEHRHQTQGHRKEEDEDESDEDDHHVSRHGRQGYEEEEDDDDDDGDDDSTEHVHQAHRHRDHEHKDDEDDSEEDYHHVPGVLRIALSTASGAAAAYSAPCLNFPISTNTPFTLVWSLREQNRS

SEQ ID NO: 9 - protein, 84 aa, Artificial Sequence
  Description of Artificial Sequence Synthetic peptide
MHSCNEHPMHLHRPHLHHMHSHHPMGHHSHGHHLHGHHPHSHHLGHHPFGHHPHLHHPHLHHPHGHHPHFHHPHFHDFLDHHHH

SEQ ID NO: 10 - DNA, 35 bp, Artificial Sequence
  Description of Artificial Sequence Synthetic primer
ggtattgagggtcgcgtggcaaaaaacattcaagc

SEQ ID NO: 11 - DNA, 37 bp, Artificial Sequence
  Description of Artificial Sequence Synthetic primer
agaggagagttagagccttaacccagtaacgtgcgca

SEQ ID NO: 12 - DNA, 1679 bp, Escherichia coli
gatatgatcgaccagctggaagcacgcattcgtgcgaaagccagtcagctggacgaagcgcgtcgaattgacgttcagcaggttgaaaaataataacgtgatgggaagcgcctcgcttcccgtgtatgattgaacccgcatggctcccgaaacattgagggaagcgttgagggttcatttttatattcagaaagagaataaacgtggcaaaaaacattcaagccattcgcggcatgaacgattacctgcctggcgaaacggccatctggcagcgcattgaaggcacactgaaaaacgtgctcggcagctacggttacagtgaaatccgcttgccgattgtagagcagaccccgctattcaaacgtgcgattggtgaagtcaccgacgtggttgaaaaagagatgtacacctttgaggatcgcaatggcgacagcctgactctgcgccctgaagggacggcgggctgtgtacgcgccggcatcgagcatggtcttctgtacaatcaggaacagcgtctgtggtatatcgggccgatgttccgtcacgagcgtccgcagaaagggcgttatcgtcagttccatcagttgggctgcgaagttttcggtctgcaaggtccggatatcgacgctgaactgattatgctcactgcccgctggtggcgcgcgctgggtatttccgagcacgtaactcttgagctgaactctatcggttcgctggaagcacgcgccaattaccgcgatgcgctggtggcattccttgagcagcataaagaaaagctggacgaagactgcaaacgccgcatgtacactaacccgctgcgcgtgctggattcaaaaaatccggaagtgcaggcgcttctcaacgacgctccggcattaggtgactatctggacgaggaatctcgtgagcattttgccggtctgtgcaaactgctggagagcgcggggatcgcttacaccgtaaaccagcgtctggtgcgtggtctggattactacaaccgtaccgttttcgagtgggtgactaacagtctcggctcccagggcaccgtgtgtgcaggcggtcgttatgacggtcttgtggaacaactgggcggtcgtgcaacaccggctgtcggttttgctatgggcctcgaacgtcttgtattgttagtacaggccgttaatccggaatttaaagccgatcctgttgtcgatatatacctggtggcttcaggtgctgatacacaatctgcggctatggcattagctgagcgtctgcgtgatgaattaccgggcgtgaaattgatgaccaaccacggcggcggcaactttaagaaacagtttgcccgtgctgataaatggggtgcccgcgttgctgtggtgctgggtgagtctgaagtggctaacggcacagcagtagtgaaggatttgcgctctggtgagcaaacggcagttgcgcaggatagcgtagccgcgcatttgcgcacgttactgggttaaggaaggagaaggacagcgtggaaatttacgagaacgaaaacgaccaggtagagcggttaaacgcttttttgctgaaaatggcaaagcactggctgttggggtgattttggcgttggcgcactgattggctggcgctactggaacagccatcaggttgattctgcacgctccgcttctcttgcctatcaaaatgcggttacc

SEQ ID NO: 13 - protein, 424 aa, Escherichia coli
MAKNIQAIRGMNDYLPGETAIWQRIEGTLKNVLGSYGYSEIRLPIVEQTPLFKRAIGEVTDVVEKEMYTFEDRNGDSLTLRPEGTAGCVRAGIEHGLLYNQEQRLWYIGPMFRHERPQKGRYRQFHQLGCEVFGLQGPDIDAELIMLTARWWRALGISEHVTLELNSIGSLEARANYRDALVAFLEQHKEKLDEDCKRRMYTNPLRVLDSKNPEVQALLNDAPALGDYLDEESREHFAGLCKLLESAGIAYTVNQRLVRGLDYYNRTVFEWVTNSLGSQGTVCAGGRYDGLVEQLGGRATPAVGFAMGLERLVLLVQAVNPEFKADPVVDIYLVASGADTQSAAMALAERLRDELPGVKLMTNHGGGNFKKQFARADKWGARVAVVLGESEVANGTAVVKDLRSGEQTAVAQDSVAAHLRTLLG

(19) United States
(12) Patent Application Publication
(10) Pub. No.: US 2005/0287190 A1
(43) Pub. Date: Dec. 29, 2005

(54) Methods of suppressing endotoxin effects in animal feeds containing E. coli biomass

(75) Inventors: Mervyn L. de Souza, Plymouth, MN (US); Michael A. Messman, Becker, MN (US)

Correspondence Address: CARGILL, INCORPORATED, LAW/24, 15407 MCGINTY ROAD WEST, WAYZATA, MN, 55391, US

(73) Assignees: Cargill, Incorporated; CAN Technologies, Inc.

(21) Appl. No.: 11/138,642

(22) Filed: May 26, 2005

Related U.S. Application Data
(60) Provisional application No. 60/575,272, filed on May 28, 2004.
(60) Provisional application No. 60/578,092, filed on Jun. 8, 2004.

(51) Int. Cl. C12N 1/21; A23K 1/165

(52) U.S. Cl. 424/442; 435/252.33

(57) ABSTRACT

Disclosed are compositions and methods for supplementing ruminant feeds. The compositions generally have an enhanced histidine content, e.g., compositions which include at least one ingredient that has an enhanced content of histidine. The histidine-enriched ingredient is commonly derived from a non-animal source. The methods can include feeding ruminants feed compositions that include the histidine-enriched ingredient to improve milk production. In some embodiments, the feed composition includes an E. coli. biomass derived from fermentation of E. coli, wherein the biomass includes (a) an inhibitor of at least one of the endotoxic effects of lipopolysaccharides of E. coli; and (b) histidine, at least one histidine-rich protein or a mixture thereof.

### CROSS-REFERENCE TO RELATED APPLICATIONS

The present application claims the benefit of U.S. provisional application No. 60/575,272, filed May 28, 2004; and U.S. provisional application No. 60/578,092, filed Jun. 8, 2004, the entire contents of which are incorporated by reference herein in their entireties.

### BACKGROUND

All animals require amino acids (AA), the building blocks of proteins necessary for optimal growth, reproduction, lactation, and maintenance. Amino acids absorbed in the cow's small intestine are derived from microbial protein and from dietary proteins that are undegraded in the rumen. Proteins digested in the small intestine must supply 10 essential amino acids (EAA), which cannot be manufactured by the cow, including arginine, histidine, isoleucine, leucine, lysine, methionine, phenylalanine, threonine, tryptophan, and valine. Ideally, the relative proportions of each of the EAA absorbed would exactly match the cow's requirements, because a shortage of one can limit the utilization of others.

Ruminants (cattle, sheep) complicate protein nutrition because they have pre-stomach chambers where digestion occurs. In the first two chambers, the rumen and the reticulum, a population of symbiotic bacteria and protozoa ferment the feeds and grow from non-protein nitrogen sources like ammonia or urea. These bacteria can digest fiber in plants enabling cattle to obtain energy from these feeds. They also synthesize protein from inexpensive byproducts. Microbial protein production is directly related to microbial growth, which is largely determined by the presence of carbohydrates such as starch, non-detergent fiber (NDF), sugars, and residual non-fiber carbohydrates (e.g., pectin and beta-glucans). The microbial population continuously washes out of the rumen to the true stomach (i.e., abomasum) where it is digested to supply amino acids to the cow.

In addition to obtaining amino acids from microbial produced protein, ruminants also obtain amino acids from undegraded essential amino acids (UEAA) that pass from the rumen to the abomasum. Lactating ruminants excrete more of certain amino acids in milk, (e.g., histidine) than are consumed in the diet and appear at the small intestine of the cow. These amino acids that are in deficit are called limiting amino acids. Supplementation of limiting amino acids to the animal will improve milk production and milk component composition. Limiting amino acids may be provided in the form of UEAA.

Amino acids may be produced in fermentation systems. However, many fermentation systems use bacteria that include lipopolysaccharides that are known to be endotoxic. Lipopolysaccharides (LPS), or endotoxins, are major structural and functional components of the outer membrane of gram-negative bacteria. These macromolecules exhibit a variety of toxic and proinflammatory activities and are causally related to the pathogenesis of gram-negative sepsis and septic shock . Many of the local and systemic pathophysiologic phenomena produced by LPS in the exposed host result from the ability of LPS to activate host inflammatory cells.

### SUMMARY

Compositions and methods directed generally to increasing milk production in dairy cattle and other ruminants are provided herein. Feeding ruminant animals for optimum production of animal products involves understanding amino acid, fatty acid, and carbohydrate nutrition. Compositions and methods of improving the amino acid nutrition of ruminant animals are provided herein. Also provided herein is a method to alleviate amino acid limitation and improve milk production and milk component composition of lactating ruminants by feeding ruminants a feedstuff that has an enhanced content of one or more limiting amino acids.

By feeding the cattle a particular feed composition which delivers an improved balance of the ten essential amino acids, the cow's milk production may be increased. In particular, the feed composition may have an enhanced content of one or more limiting amino acids, as determined by the cow's amino acid requirements for maintenance, growth, and milk production. Limiting amino acids may include histidine, lysine, methionine, phenylalanine, and threonine. The feed composition may be formulated to deliver an improved balance of essential amino acids post-ruminally.

Histidine-rich protein II from the malaria parasite, Plasmodium falciparum, has been shown to inhibit the ability of LPS to evoke an inflammatory response in CD14-transfected THP-1 cells. (See Bosshart et al., FEBS Lett. 2003 Oct. 9; 553(1-2):135-40.) Further, a short synthetic peptide derived from human histidine- and proline-rich glycoprotein has also been shown to exhibit LPS-inhibitory effects in CD14-transfectants. (See Iwagaki et al., Infect Immun. 2000 March; 68(3):1655-63 and Bosshart et al., Infect Immun. 2002 December; 70(12):6904-10.) Taken together, these observations demonstrate the capacity of histidine-rich peptides to neutralize LPS-induced proinflammatory host responses.

Also described herein is an E. coli. biomass produced by an E. coli. fermentation process, which includes an inhibitor of at least one of the endotoxic effects of lipopolysaccharides of E. coli., and at least one histidine-rich protein or histidine. The histidine-rich protein may be an inhibitor of at least one of the endotoxic effects of lipopolysaccharides of E. coli. Also described herein is a method of inhibiting the endotoxic properties of an E. coli biomass by expressing at least one inhibitor of the endotoxic effect of lipopolysaccharides in E. coli. The inhibitor may include a histidine-rich protein. In one embodiment, the inhibitor is co-expressed with at least one other amino acid or protein, which may be histidine and a histidine-rich protein respectively. An E. coli biomass produced by such a method may be safe for animal feed. This may result in cost savings based on elimination of down stream purification of histidine or histidine-rich proteins, losses of histidine or histidine-rich proteins in the biomass during separation of the fermentation broth from the biomass, and costs associated with disposal of the biomass.

The feed composition typically includes at least one ingredient that has an enhanced content of histidine that is derived from a non-animal source (e.g., a bacteria, yeast, and/or plant). For example, the composition may include a histidine source which includes L-His and a biomass formed during fermentation of a histidine-producing microorganism and at least one additional nutrient component. In another example, the feed composition includes histidine source which includes L-His and dissolved and suspended constituents from a fermentation broth formed during fermentation of a histidine-producing microorganism and at least one additional nutrient component. In a further embodiment, the feed composition has a crude protein fraction which includes at least one histidine-rich protein of non-animal origin.

The composition may be used in several forms including, but not limited to, complete feed form, concentrate form, blender form and base mix form. Feed forms for increasing milk production in diary cattle by balancing the essential amino acids via a particular complete feed, concentrate, or blender or base mix form of the composition are described in U.S. Pat. No. 5,145.695 and U.S. Pat. No. 5,219,596, the disclosures of which are incorporated by reference herein in their entireties.

If the composition is in the form of a complete feed, the percent protein level, (crude protein content) may be about 10 to about 25 percent, more suitably about 14 to about 24 percent; whereas, if the composition is in the form of a concentrate, the protein level may be about 30 to about 50 percent, more suitably about 32 to about 48 percent. If the composition is in the form of a blender, the protein level in the composition may be about 20 to about 30 percent, more suitably about 24 to about 26 percent; and if the composition is in the form of a base mix, the protein level in the composition may be about 55 to about 65 percent. Unless otherwise stated herein, percentages are stated on a weight percent basis.

The complete feed form composition generally contains one or more ingredients such as wheat middlings (“wheat mids”), corn, soybean meal, corn gluten meal, distillers grains or distillers grains with solubles, salt, macro-minerals, trace minerals and vitamins. Other potential ingredients may commonly include, but not be restricted to sunflower meal, malt sprouts and soybean hulls.

The concentrate form composition generally contains wheat middlings, corn, soybean meal, corn gluten meal, distillers grains or distillers grains with solubles, salt, macro-minerals, trace minerals and vitamins. Alternative ingredients would commonly include, but not be restricted to sunflower meal and malt sprouts. The blender form composition generally contains wheat middlings, corn gluten meal, distillers grains or distillers grains with solubles, salt, macro-minerals, trace minerals and vitamins. Alternative ingredients would commonly include, but not be restricted to, corn, soybean meal, sunflower meal, malt sprouts and soybean hulls.

The base form composition generally contains wheat middlings, corn gluten meal, and distillers grains or distillers grains with solubles. Alternative ingredients would commonly include, but are not restricted to, soybean meal, sunflower meal, malt sprouts, macro-minerals, trace minerals and vitamins.

The complete feed form composition, concentrate form composition, blender form composition, and base form composition also include a product that has an enhanced amino acid content with regard to one or more selected amino acids. In particular, the product may have an enhanced amino acid content with regard to one or more limiting amino acids for milk production. The product may have an enhanced amino acid content because of the presence of free amino acids in the product and/or the presence of proteins or peptides that include the amino acid in the product. For example, the product may have an enhanced content of histidine present as free amino acids and/or present in histidine-rich proteins. Typically, the product is derived from a non-animal source such as microorganisms (e.g., bacteria and yeast) and/or plants.

The product may have an enhanced content of one or more amino acids, in particular, one or more essential amino acids determined to be limiting for milk production. Limiting amino acids may include histidine, lysine, methionine, phenylalanine, threonine, isoleucine, and/or tryptophan, which may be present in the product as a free amino acid or as a protein or peptide that is rich in the selected amino acid. For example, the product may include at least one histidine-rich proteins. A histidine-rich protein will typically have at least 5% histidine residues per total amino acid residues in the protein, and more typically, at least 10% histidine residues per total amino acid residues in the protein. A product with an enhanced content of histidine, typically has a histidine content (including free histidine and histidine present in a protein or peptide) of at least 3.0 wt. % relative to the weight of the crude protein and amino acid content of the product, and more suitably at least 5.0 wt. % relative to the weight of the crude protein and amino acid content of the product.

A product with an enhanced content of histidine may be produced in a microbial fermentation process. In one example, a bacteria that overproduces histidine is grown in a fermentation system and the fermentation broth and/or fermentation biomass are further processed to produce a product that has an enhanced content of histidine. The fermentation broth or biomass may be dried (e.g., spray-dried), to produce the product with an enhanced content of histidine.

Histidine or a product having an enhanced content of histidine may be at least partially purified from the fermentation broth or lysed biomass. For example, histidine or histidine-rich proteins may be isolated based on the isoelectric point of histidine. Histidine may be isolated based on the presence of an imidazole moiety in the molecule. Similarly, the presence of the histidine in a histidine-rich protein may be used to isolate the protein, based on the isoelectric point of the protein. The desired isoelectric point for a histidine-rich protein may be varied by using recombinant technology to alter the amino acid composition of the protein (e.g., to create a protein having a selected histidine content).

The unique isoelectric point (pI) of histidine compared to other amino acids may permit selective precipitation of histidine, preferential extraction into organic solvents, and binding to various ion exchange resin or metal chelation matrices. A stretch of six (6) histidine residues is called a histidine tag, which binds to transition metals such as nickel (Ni) and may be used to facilitate isolation of the protein (e.g., by binding the protein to a nickel-containing matrix). Other transition metals may be used, such as copper (Cu). In addition, the imidazole moiety of histidine may permit the use of unique combinations of size exclusion chromatography and ion-exchange resins to isolate histidine from fermentation broth containing other amino acids and by-products. Additionally, the unique pI of histidine could result in specific and unique pI values for histidine-rich proteins thus permitting selective precipitation of these proteins from other cellular proteins for subsequent use in feed or food.

Histidine-rich proteins may be selected from those histidine-rich proteins described in the literature, such as the histidine-rich protein II from Plasmodium falciparum and one or more of the proteins from class of proteins called “histatins,” which demonstrate anti-bacterial and anti-fungal activities. A histidine-rich protein may also comprise specific fragments of known histidine-rich proteins that have an increased histidine content compared to the full-length protein. For example, the histidine-rich protein II from Plasmodium falciparum has a histidine composition of about 32%. The fragment of this protein from amino acid 61 to 130 has a histidine composition of about 44%. The fragment of this protein from amino acid 58 to 80 has a histidine composition of about 55%. A histidine-rich protein does not need to retain its native function to be suitable for the compositions or methods described herein.

In addition, histidine-rich proteins may be in the form of recombinantly-engineered proteins. For example, as noted above, poly-histidine motifs called “histidine tags” are commonly added to proteins to aid in purification because poly-histidine motifs bind to transition metals such as nickel. The recombinantly-engineered proteins may have an enhanced content of other amino acids in addition to histidine. In particular, the proteins may have an enhanced content of one or more of the essential amino acids, or the proteins may have an enhanced content of one or more of the other limiting amino acids for milk production, which may include lysine, methionine, phenylalanine, threonine, isoleucine, and tryptophan. As such, the recombinantly-engineered proteins may be designed to include a selected profile of amino acids. The ratios of the amino acids in the recombinantly-engineered proteins may be varied or designed to match the ratios that are predicted to be optimal for dairy cattle based on feeding studies or predictions. In one embodiment, the selected profile of amino acids, e.g., in a recombinantly produced protein, is similar to the profile of blood meal. After a protein has been designed and its gene has been cloned into an expression vector, the protein may be expressed (or over expressed) in a microbial host such as E. coli. Corynebacterium, Brevibacterium, Bacillus, Yeast, etc.

In order to optimize the expression of the protein in the host, the sequence of the protein may be selected to utilize specific tRNAs that are prevalent in the host. Alternatively, selected tRNAs may be co-expressed in the host to facilitate expression of the protein.

The recombinantly-engineered proteins may include specific sequences to facilitate purification of the proteins. For example, the proteins may include histidine tags. The proteins may also include “leader sequences” that target the protein to specific locations in the host cell such as the periplasm, or target the protein for secretion.

The recombinantly-engineered proteins may also include protease cleavage sites to facilitate cleavage of the proteins in the abomasum and enhance delivery of amino acids in the protein to the small intestine. For example, one such protease is pepsin, one of the protein-digesting enzymes of the abomasum in cattle. Pepsin demonstrates a preferential cleavage of peptides at hydrophobic, preferentially aromatic, residues in the P1 and P1′ positions. In particular, pepsin cleaves proteins on the carboxy side of phenylalanine, tryptophan, tyrosine, and leucine.

In another example, histidine-rich proteins may be augmented with peptides or proteins that have an enhanced content of other amino acids, in particular limiting amino acids. For example, a product may include one or more proteins that have an enhanced content of one or more of the same or different amino acids. As such, the product may include multiple proteins, peptides, and/or amino acids.

The histidine-rich proteins or peptides may be over-expressed in a microbial host such as a species of Eschrichia, Corynebacterium, Brevibacterium, Bacillus, Yeast, etc. The entire microbial biomass may be spray-dried and used in the animal feed or the histidine-rich proteins and related proteins or peptides may be at least partially purified from the biomass. Alternatively, where the microbial host excretes histidine and/or a histidine-rich protein, the histidine-enriched broth may be separated from the biomass produced by the fermentation and the clarified broth may be used as an animal feed ingredient, e.g., either in liquid form or in spray dried form. In one embodiment, histidine-rich proteins may be purified by binding histidine tags in the proteins to a matrix that includes nickel metal.

It may be desirable to use microbial hosts that do not contain lipopolysaccharides (“LPS”) that have endotoxic effects, for example a Gram-positive bacteria such as Corynebacteria and Brevibacterium. Gram-negative bacteria, such as E. coli, often include LPS that have an endotoxic effect. Selection of a bacteria that does not include endotoxic LPS may be particularly important when a biomass is to be prepared and used as histidine source, because the majority of LPS remain associated with bacteria and are not released substantially into the fermentation broth unless the bacteria are lysed. As such, endotoxic LPS would be expected to be localized within the biomass after fermentation.

The histidine-rich proteins may be further treated to facilitate rumen bypass. For example, the histidine-rich proteins may be coated with polymeric compounds, formalized protein, fat, mixtures of fat and calcium, mixtures of fat and protein, and with metal salts of long chain fatty acids. In particular, the histidine-rich proteins may be coated with a mixture of a metal salt of a fatty acid (e.g., zinc stearate) and a fatty acid (e.g., stearic acid). The histidine-rich proteins may also be coated with pH-sensitive polymers pH-sensitive polymers. A pH-sensitive polymer is stable at ruminal pH, but breaks down when it is exposed to abomasal pH, releasing the protein for digesting in the abomasums and absorption in the small intestine. Similarly, free amino acids may be of little value in ruminant diets because they are degraded rapidly in the rumen. As such, free amino acids may be coated to provide protection from degradation in the rumen.

In one aspect, the disclosed method includes several steps. First, an amino acid or a protein that is rich in one or more amino acids is synthesized. As noted above, a suitable amino acid may be histidine and a suitable protein may be a histidine-rich protein. The amino acid and/or amino acid-rich protein may be synthesized using a microbial fermentation system to produce a fermentation biomass, which may be dried (e.g., spray-dried) to provide a dried fermentation biomass. Alternatively, the amino acid and/or protein may be present in the fermentation broth, which may be separated from the fermentation biomass (e.g., via filtration) and spray-dried to produce a dried fermentation broth that has an enhanced content of the amino acid and/or protein. Further, the amino acid and/or amino acid-rich protein may be isolated or at least partially purified from either the biomass and/or broth prior to preparing a dried product. The dried fermentation biomass, dried fermentation broth, and/or dried product may be coated with a coating to provide a coated product. The coating protects the product and enables it to pass through the rumen with reduced degradation and to deliver the product to the small intestine. As such, the coating allows the coated products to bypass the rumen, (i.e., allows rumen bypass). The coated product may be fed to a ruminant to improve milk production as well as to improve milk protein composition.

### BRIEF DESCRIPTION OF THE DRAWINGS

FIG. 1 is a schematic representation of a model for microbial growth. NDF—“neutral detergent fiber”; NFC—“non-fiber carbohydrates”; VFA—“volatile fatty acids”; RDP—“rumen degradable protein”; rH—“pH of the rumen”.

FIG. 2 is a schematic representation of a typical spin disk process for encapsulating products.

### DETAILED DESCRIPTION

Histidine is considered to be a primary rate limiting amino acid in ruminant feed and its concentration in feed is directly correlated to milk production in dairy cows. Blood meal is currently used in animal feed and is a rich source of histidine. Replacements for blood meal lack a similar histidine content and a feed lacking blood meal would need to be supplemented with histidine to fulfill amino acid requirements. In addition, as milk yields increase there is a corresponding increase in amino acid requirements in addition to histidine. This increase in amino acid requirements needs to be met as well.

Protein must escape ruminal degradation and pass to the small intestine to supply sufficient amounts of amino acids. The primary methods developed to prevent fermentative digestion of amino acids include (1) structural manipulation of the amino acid to produce amino-acid analogs that demonstrate reduced degradation in the rumen and (2) coating a product that has an enhanced amino acid content with a composition that protects the product from degradation in the rumen. Single histidine residues are more readily degraded in the rumen than histidine present in proteins or peptides, and as such, histidine-rich proteins may provide an advantage over single histidine residues. In addition to providing a source of histidine for ruminant feed, histidine-rich proteins may closely resemble the “histidine rich” proteins that are present in blood meal.

Histidine-rich proteins are known from the literature and include the histidine-rich protein II from Plasmodium falciparum, Accession No. AAC47453, which has a histidine content of more than 32% (histidine residues/total residues) and the amino acid sequence:

| | |
|---|---|
| 1 | mvsfsknkvl saavfasvll ldnnnsafnn nlcsknakgl nlnkrllhet |
| 51 | qahvddahha hhvadahhah haadahhahh aadahhahha adahhahhaa |
| 101 | dahhahhaay ahhahhaada hhahhasdah haadahhaay ahhahhaada |
| 151 | hhahhasdah haadahhaay ahhahhaada hhaadahhat dahhahhaad |
| 201 | arhatdahha adahhatdah haadahhaad ahhatdahha adahhatdah |
| 251 | haadahhaad ahhatdahha hhaadahhaa ahhatdahha tdahhaaahh |
| 301 | eaathclrh |

Another histidine-rich protein is the histidine-rich glycoprotein from Mus muculus, Accession No. AAH11168, which has a histidine content of more than 10% (histidine residues/total residues) and the amino acid sequence:

| | |
|---|---|
| 1 | mkvlttalll vtlqcshals ptncdasepl aekvldlink grrsgyvfel |
| 51 | lrvsdahldr agtatvyyla ldviesdcwv lstkaqddcl psrwqseivi |
| 101 | gqckviatry snesqdlsvn gyncttssvs salrntkdsp vlldffedse |
| 151 | lyrkqarkal dkyktdngdf asfrveraer virarggert nyyvefsmrn |
| 201 | cstqhfprsp lvfgfcrall sysietsdle tpdsidince vfniedhkdt |
| 251 | sdmkphwghe rplcdkhlck lsgsrdhhht hktdklgcpp ppegkdnsdr |
| 301 | prlqegalpq lppgypphsg anrthrpsyn hscnehpchg hrphghhphs |
| 351 | hhppghhshg hhphghhphs hhshghhppg hhphghhphg hhphghhphg |
| 401 | hhphghdfld ygpcdppsns qelkgqyhrg ygpphghsrk rgpgkglfpf |
| 451 | hhqqigyvyr ippinigevi tlpeanfpsf slpncnrslq peiqpfpqta |
| 501 | srscpgkfes efpqiskffg ytppk |

Another histidine-rich protein is the actinorizal nodulin AgNOD-GHRP from Alnus glutinosa, Accession No. AAD00171, which has a histidine content of approximately 15% (histidine residues/total residues) and the amino acid sequence:

| | |
|---|---|
| 1 | mgysktflhl glafavvlli ssdvsasela vaaqtkenmq tdgveedkyh |
| 51 | ghrhvhghgh ghvhgngneh ghghhhgrgh pghgaaadet etetetnqn |

Another histidine-rich protein is human histidine-rich calcium-binding protein, precursor, Accession No. AAH69795, which has a histidine content of approximately 12% (histidine residues/total residues) and the amino acid sequence:

| | |
|---|---|
| 1 | mghhrpwlha svlwagvasl llppamtqql rgdglgfrnr nnstgvagls |
| 51 | eeasaelrhh lhsprdhpde nkdvstengh hfwshpdrek ededvskeyg |
| 101 | hllpghrsqd hkvgdegvsg eevfaehggq arghrghgse dtedsaehrh |
| 151 | hlpshrshsh qdededevvs sehhhhulrh ghrghdgedd egeeeeeeee |
| 201 | eeeeasteyg hqahrhrghg seededvsdg hhhhgpshrh qgheeddddd |
| 251 | dddddddddd dvsieyrhqa hrhqghgiee dedvsdghhh rdpshrhrsh |
| 301 | eeddnddddv steyghqahr hqdhrkeeve avsgehhhhv pdhrhqghrd |
| 351 | eeededvste rwhqgpqhvh hglvdeeeee eeitvqfghy vashqprghk |
| 401 | sdeedfqdey ktevphhhhh rvpreedeev saelghqaps hrqshqdeet |
| 451 | ghgqrgsike mshhppghtv vkdrshlrkd dseeekekee dpgsheedde |
| 501 | sseqgekgth hgsrdqedee deeeghglsl nqeeeeeedk eeeeeeedee |
| 551 | rreeraevga plspdhseee eeeeegleed eprftiipnp ldrreeagga |
| 601 | sseeesgedt gpqdaqeygn yqpgslcgyc sfcnrctece schcdeenmg |
| 651 | ehcdqcqhcq fcylcplvce tvcapgsyvd yfssslyqal admletpep |

Other histidine-rich proteins include the class of proteins called “histatins.” Histatins are histidine-rich proteins which occur in saliva and have anti-fungal and anti-bacterial properties. See Neuman et al., (1996) Electrophoresis 17: 266-270. These histidine-rich proteins or peptides may be used as a histidine source in animal feed, for example animal feed for dairy cattle. Because histatins have anti-fungal and anti-bacterial properties, in addition to serving as a histidine source, histatins may provide animal feed with a longer shelf life.

Animal feeds which include an E. coli. biomass and/or dissolved and suspended constituents from a fermentation broth produced by an E. coli. fermentation process, which includes an inhibitor of at least one of the endotoxic effects of lipopolysaccharides of E. coli are also provided in certain embodiments herein. The animal feed may include as one source of protein at least one histidine-rich protein and/or histidine which has been produced by an E. coli. fermentation process. Such a protein source may suitably include one or more histidine-rich proteins which are an inhibitor of at least one of the endotoxic effects of lipopolysaccharides of E. coli. Also described herein is a method of inhibiting the endotoxic properties of an E. coli biomass by expressing at least one inhibitor of the endotoxic effect of lipopolysaccharides in E. coli. The inhibitor may include a histidine-rich protein. In one embodiment, the inhibitor may be co-expressed with at least one other amino acid and/or protein, which may be histidine and a histidine-rich protein respectively. An E. coli biomass produced by such a method may be sufficiently safe to be used for animal feed. This may result in cost savings based on elimination of down stream purification of histidine or histidine-rich proteins, losses of histidine or histidine-rich proteins in the biomass during separation of the fermentation broth from the biomass, and costs associated with disposal of the biomass.

Examples of histidine-rich proteins, which are inhibitors of at least one of the endotoxic effects of lipopolysaccharides of E. coli, include poly-L-histidine, histidine-rich protein II from the malaria parasite, Plasmodium falciparum, and/or histidine-rich fragments from human histidine- and proline-rich glycoprotein. Synthetic peptides having related sequences (e.g., at least about 80% and more suitably at least about 90% amino acid homology) and similar histidine content to such proteins and/or histidine-rich fragments thereof may also be included in the present animal feed compositions.

Amino Acid Demand. Limiting amino acids may be supplied to an animal to increase production of a chosen animal product (e.g., milk) by supplementing the animal's feed with the limiting amino acid. Limiting amino acids may be identified by analyzing the amino acid profile of the chosen animal product (i.e., output profile) and comparing this profile to the profile of amino acids supplied to the animal (i.e., input profile). Methods for determining amino acid requirements are known in the art and are described in U.S. Pat. No. 5,145,695 and U.S. Pat. No. 5,219,596, which are incorporated by reference herein in their entireties.

For example, the amino acid profile of milk can be compared to the profile of amino acids produced by microbes within the digestive tract of the animal (i.e., microbial amino acid profile). Differences between the microbial and milk amino acid profiles indicate where amino acids may be in excess or limiting. However, this amino acid profile comparison provides only part of the needed information in order to increase production of a chosen animal product. The efficiency with which the body incorporates amino acids in the small intestine into a chosen animal product must also be considered. By determining the output/input amino acid profile ratio and by determining the efficiency of incorporation, dairy digestible amino acid requirements may be determined. It has been established that histidine, lysine, methionine, phenylalanine, and threonine are likely to be limiting amino acids for milk production in dairy cows. A similar determination may be performed for the amino acid profile of muscle.

Supply of Amino Acids. Ruminants derive amino acids from two sources: (1) microbial protein as determined by microbial growth; and (2) protein that remains undegraded in the rumen (i.e., “rumen undegraded protein” or “RUP”). Microbial growth may be predicted based on the carbohydrates available for fermentation in the rumen (e.g., starch, sugar, neutral detergent fiber, pectin, and beta-glucan), the supply of rumen degradable protein, and pH of the rumen. Because microbial proteins are not fully digestible, the supply of microbial amino acids supplied by the microbial protein must be adjusted based on the digestibility of the protein to provide a digestable microbial amino acid value.

The second source of amino acids is feed ingredients that remain undegraded after passing from the rumen to the abomasum (i.e., the bypass protein fraction). Amino acids within a feed ingredient are processed and utilized (i.e., degraded) by microbes in the rumen at different rates. As such, different amino acids will have different undegradable essential amino acid (“UEAA”) values. In addition, a UEAA value may be adjusted based on the digestability of an amino acid in the small intestine to provide a digestible UEAA value. The sum of digestible microbial amino acids and digestible UEAA's is the digestible amino acid contribution that will be provided to the small intestine.

In diet formulation, the predicted digestible microbial amino acid contribution from rumen fermentation is subtracted from the animal's amino acid requirements, as determined by the animal's profile. The amounts of amino acids that need to be supplied as UEAA's from feed are the difference between the animal's amino acid requirements and the amino acids supplied from digestible microbial amino acids.

Synthesis of histidine-rich products. Histidine-rich products may include products that have an enhanced content of histidine as a free amino acid and/or products that include histidine-rich proteins. Histidine-rich products may be produced by methods known in the art. For example, a histidine-rich fermentation broth may be used as a source of histidine. The histidine-rich fermentation broth may be produced by single-cell organisms (e.g., microorganisms such as bacteria or yeast) that are selected or engineered to overproduce histidine. Suitable microorganisms may include microorganisms belonging to the genus Eschrichia, Bacillus, Microbacterium, Arthrobacter, Serratia, and Corynebacterium. Gram-negative bacteria are known to produce lipopolysaccharides (“LPS”), which are endotoxins. As such, it may be desirable to select a Gram-positive bacteria as the host-cell, (e.g., Corynebacteria and Brevibacteria), particularly when a biomass is to be prepared. The majority of LPS remain associated with the host-cell and are not released into the fermentation broth until the host-cell is lysed. As such, Gram-negative bacteria such as E. coli. may be suitable for producing a histidine broth.

The histidine-rich fermentation broth may be spray-dried and used directly as a histidine source or the broth may be concentrated. In another embodiment, histidine may be at least partially purified from the fermentation medium and biomass. The microbial produced histidine may then be prepared based on rumen bypass technology and added to feed at the required level.

Alternatively, microbes may be engineered to accumulate and retain histidine and the microbes may be prepared as a spray-dried biomass product. Optionally, the biomass may be separated by known methods, such as separation, decanting, a combination of separation and decanting, ultrafiltration or microfiltration. The biomass product may be further treated to facilitate rumen bypass. In one embodiment, the biomass product may be separated from the fermentation medium, spray-dried, and optionally coated to facilitate rumen bypass, and added to feed as a histidine source.

In a further embodiment, microbes may be engineered to produce histidine-rich proteins. Histidine-rich proteins may include known and characterized proteins (e.g., histidine-rich protein II of Plasmodium falciparum and or histatins) and engineered proteins (e.g., proteins designed to have a selected amino acid profile.) For example, histidine-rich protein II of Plasmodium falciparum or a selected histatin may be cloned into an expression vector and introduced into a suitable host cell. Alternatively, an recombinantly engineered protein that has a chosen amino acid profile may be cloned into an expression vector and introduced into a suitable host cell (e.g., microbe).

The histidine-rich proteins may be secreted into the fermentation media, or alternatively, the histidine-rich proteins may accumulate in the microbes. The microbes may be prepared as a spray-dried biomass product, or the histidine-rich proteins or peptides may be isolated from the microbial biomass to provide a histidine-rich product. In either case, the histidine-rich product may be further treated to enhance rumen bypass. The treated product then may be added to feed as a histidine source.

In addition to producing histidine-rich products in fermentation systems, histidine-rich products also may be produced in transgenic plant systems. Methods for producing transgenic plant systems are known in the art.

Rumen protection of histidine and histidine-rich products. Histidine and/or histidine-rich products (i.e., ingredients) may be coated or encapsulated to decrease degradation in the rumen (i.e., to facilitate rumen bypass). A suitable coating may have a relatively high melting temperature as described below.

Suitable coatings may include a mixture of a hydrophobic, high melting point compound and a lipid. The combination of one or more, hydrophibic, high melting point compounds (e.g., mineral salts of fatty acids such as commercial grade zinc stearate) with one or more type of lipid, forms a coating compound that can protect the content and functionality of the coated ingredient(s). These coatings can be formulated to meet the needs of high temperature and pressure processing conditions as well as protection of the amino acid payload from the microbial environment of the rumen. Suitable coatings are described in U.S. Patent Publication No. 2003/0148013, which is incorporated herein by reference in its entirety.

Hydrophobic, high melting point compounds typically have a melting point of at least about 70° C., and more desirably, greater than 100° C. In particular, zinc salts of fatty acids, which have a melting point between about 115° C. and 130° C., are suitable hydrophobic, high melting point compounds.

The lipid component typically has a melting point of at least about 0° C. and more suitably no less than about 40° C. The lipid component may include vegetable oil, such as soybean oil. In other embodiments, the lipid component may be a triacylglycerol with a melting point of about 45-75° C. Commercial grade stearic acid may be selected as a representative lipid from a group including but not limited to: stearic acid, hydrogenated animal fat, animal fat (e.g., animal tallow), vegetable oil, (such as crude vegetable oil and/or hydrogenated vegetable oil, either partially or fully hydrogenated), lecithin, palmitic acid, animal oils, wax, fatty acid esters (C₈ to C₂₄), fatty acids (C₈ to C₂₄).

The coating may be present in the coated product in an amount from 1-2000 wt. %, relative to the weight of the coated ingredient. Commonly, the coating represents about 25 to 85 wt. %, relative to the weight of the coated ingredient.

The coating uses one or more, hydrophobic, insoluble compounds combined with a lipid. For example, commercial grade zinc stearate is extremely hydrophobic and completely insoluble in water. The addition of commercial grade zinc stearate to the coating formula may improve the protection level of the ingredient and its functionality, significantly as compared to a lipid only coating. For example, by combining zinc stearate with a somewhat insoluble lipid such as commercial grade stearic acid, the coating compound may provide better protection from leaching (i.e., loss of the active ingredient from the coated product), when the coated product is in an aqueous medium. As such, the benefit of the present coating composition may be utilized in feeds designed for ruminants to bypass the rumen and deliver the active ingredient to the small intestine.

In addition to facilitating rumen bypass, the coating may also be useful for protecting the coated ingredients against heat and pressure experienced during the manufacturing process (pelleting and extrusion). The coating composition may be useful in all types of production processes where heat is applied and heat susceptible ingredients are used. Ingredients which may benefit from this form of protection are ingredients that are subject to heat damage or degradation, such as amino acids, proteins, enzymes, vitamins, pigments, and attractants.

In addition to protecting ingredients from heat related damage or loss there is also the need to protect ingredients to damage or loss attributable to association or chemical reaction with other ingredients. The method of encapsulation may prevent harmful association with other ingredients: As such, the method of encapsulation provides the ability to prepackage or combine ingredients in a formulation, where the ingredients would be usually packaged individually.

The coating composition may be prepared in a number of ways. Preferably, the preparation process includes making a solid solution of the zinc organic salt component and the lipid component. In one embodiment, the zinc organic salt and the lipid component may be melted until they both dissolve and form a solution. The solution may then be allowed to solidify to form a solid solution.

In addition to the zinc organic acid component and the lipid component, the coating may include other ingredients. For example, the coating may include an one or more emulsifying agents such as glycerin, polysaccharides, lecithin, gelling agents and soaps, which may improve the speed and effectiveness of the encapsulation process. Additionally, the coating may include an anti-oxidant to provide improved protection against oxidation effects. Further, the coating composition may include other components that may or may not dissolve in the process of forming the solid solution. For example, the coating composition may include small amounts of zinc oxide and other elements or compounds.

After the coating composition is prepared, it can then be used to prepare the protected ingredient. One suitable procedure for preparing the protected ingredient uses encapsulation technology, preferably microencapsulation technology. Microencapsulation is a process by which tiny amounts of gas, liquid, or solid ingredients are enclosed or surrounded by a second material, in this case a coating composition, to shield the ingredient from the surrounding environment. A number of microencapsulation processes could be used to prepare the protected ingredient such as spinning disk, spraying, co-extrusion, and other chemical methods such as complex coacervation, phase separation, and gelation. One suitable method of microencapsulation is the spinning disk method. In the spinning disk method, an emulsion and/or suspension of the active ingredient and the coating composition is prepare and gravity-fed to the surface of a heated rotating disk. As the disk rotates, the emulsion/suspension spreads across the surface of the disk to form a thin layer because of centrifugal forces. At the edge of the disk, the emulsion/suspension is sheared into discrete droplets in which the active ingredient is surrounded by the coating. As the droplets fall from the disk to a collection hopper, the droplets cool to form a microencapsulated ingredient (i.e., a coated product). Because the emulsion or suspension is not extruded through orifices, this technique permits use of a higher viscosity coating and allows higher loading of the ingredient in the coating.

The encapsulation of ingredients for use in animal feeds are described in U.S. Patent Publication No. 2003/0148013, which is incorporated herein by reference in its entirety.

Amino acids may also be chemically altered to protect the amino acid in the rumen and to increase the supply of specific amino acids provided to the abomasums and small intestine. For example, methionine hydroxyl analog (MHA®) has been used as an amino acid supplement. In addition, amino acids may be provided as amino acid/mineral chelates. Zinc-methionine and zinc-lysine complexes have been used as amino acid supplements.

From a standpoint of providing a protected product, yeast may be a particularly suitable host for expressing histidine-rich proteins and/or amino acids. A lysine-accumulating yeast has been shown to accumulate from 4 to 15% of its dry weight as lysine. The majority of the lysine is contained in vacuoles that are stable when incubated with rumen fluid, but immediately released when exposed to pepsin, one of the protein-digesting enzymes of the abomasum. Thus, this organism may be a useful host for expressing proteins and/or amino acids and providing a protected feed supplement that may increase the amount of proteins and/or amino acids available for intestinal absorption.

Feeding formulations that have an enhanced content of one or more essential amino acids. Initially, an empirical approach was taken to generate essential amino acid requirements for lactating cows. The essential amino acid composition of rumen microbial protein was compared to the essential amino acid composition of milk protein (Table 1). (The same may be done for muscle protein as an indicator of amino acid requirements for growth, maintenance and reproduction.)

TABLE 1
Essential amino acid composition of milk protein compared to
microbial protein (grams amino acid/100 grams protein).

| | Microbial | Milk | Microbial Protein/ |
|---|---|---|---|
| Amino Acid | Protein | Protein | Milk Protein |
| Arginine | 5.4 | 3.3 | 1.67 |
| Histidine | 2.3 | 2.6 | 0.88 |
| Isoleucine | 7.3 | 4.6 | 1.58 |
| Leucine | 9.4 | 9.4 | 1.00 |
| Lysine | 9.3 | 7.7 | 1.21 |
| Methionine | 2.6 | 2.5 | 1.06 |
| Phenylalanine | 5.1 | 5.3 | 0.96 |
| Threonine | 6.4 | 4.4 | 1.47 |
| Tyrosine | 1.5 | 1.4 | 1.07 |
| Valine | 7.2 | 5.7 | 1.27 |

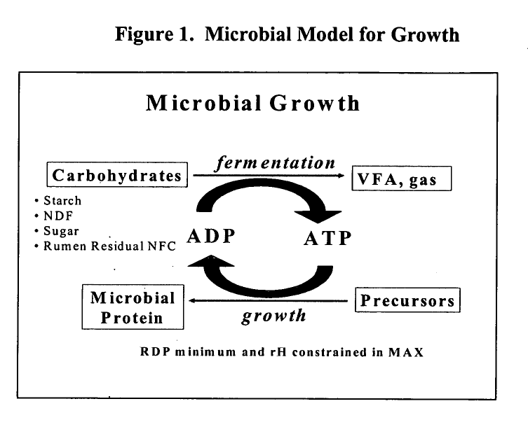

Amino acids predicted to be limiting were then candidates for further study. Once amino acid requirements were determined, a method was developed to satisfy those amino acid requirements. The first step was to account for microbial amino acid production in the rumen. A microbial model for amino acid production is provided in FIG. 1. Microbial amino acid production is determined by microbial growth, which in turn is determined by carbohydrate concentrations that are fermented in the rumen including starch, neutral detergent fiber (“NDF”), sugars, and residual non-fiber carbohydrates (“RNFC”) such as pectin and beta-glucan.

To determine the amino acid contribution of rumen microbial protein to an animal's diet, the total rumen microbial protein is multiplied by the percent of each specific amino acid present in the protein. Many researchers have found that the amino acid composition of rumen microbial protein to remain fairly constant. Digestibility of bacterial amino acids is assumed to be 80% for each amino acid. The resulting amounts of amino acids provided by rumen microbial protein were then subtracted from the amino acid requirements. The deficits, (i.e., the differences between the requirements and the amino acids supplied from rumen microbial protein), indicated the amounts of amino acids that should advantageously be supplied as undegradable essential amino acids (UEAAs) in feed.

Feed ingredients high in UEAAs (or “bypass” amino acids) were evaluated to determine potent sources of UEAAs. Blood meal has been used as a common source of UEAAs in the past. Blood meal is also a good source of histidine (Table 2).

TABLE 2
Essential amino acid composition of blood meal protein compared
to milk protein (grams amino acid/100 grams protein).

| | | | Blood Meal/ |
|---|---|---|---|
| Amino Acid | Blood Meal | Milk | Milk |
| Arginine | 3.5 | 3.3 | 1.06 |
| Histidine | 5.2 | 2.6 | 2.00 |
| Isoleucine | 1.0 | 4.6 | 0.21 |
| Leucine | 12.8 | 9.4 | 1.36 |
| Lysine | 8.4 | 7.7 | 1.09 |
| Methionine | 1.1 | 2.5 | 0.44 |
| Phenylalanine | 6.6 | 5.3 | 1.24 |
| Threonine | 4.2 | 4.4 | 0.96 |
| Tyrosine | 1.2 | 1.4 | 0.86 |
| Valine | 8.8 | 5.7 | 1.54 |

Animal amino acid requirements. Amino acids required in feeds for dairy cows are called Dairy Digestible Amino Acids (“ddAA”). The sum of the digestible microbial amino acid plus the digestible rumen undegraded essential amino acid (UEAA) concentration of that same amino acid is the ddAA. Dairy Digestible Amino Acids represent the supply of total digestible AA to the small intestine. The total amino acid requirements of a dairy animal may be determined as follows. The total amount of an amino acid required (“TAAR”) is equal to the amount required for maintenance (“Maintenance Amino Acid” or “MAA”) plus the amount of the amino acid required for milk production (“Milk Amino Acid Output” or “MAAO”) plus the amount of the amino acid required for growth (“Growth Amino Acid” or “GAA”) (i.e., TAAR=MAA+MAAO+GAA).

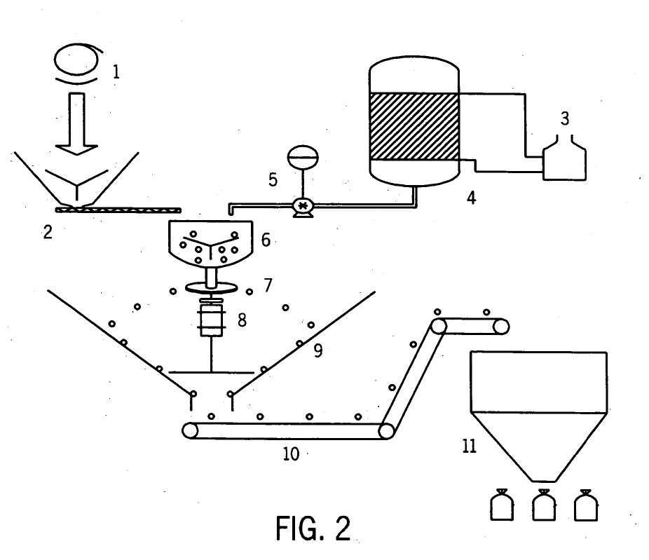

Encapsulation. The process displayed in FIG. 2 represents microencapsulation by spin disk technology. Other microencapsulation processes include spraying, centrifugal co-extrusion, and chemical means.

The process begins by preparing the coating, for example; a water-soluble nutrient may be protected from water solubility by using a fat coating. The coating is melted by heating the coating to its melting point in the fat holding tank until the coating is liquefied. The nutrient is typically a dry powder of an an amino acid, biomass, peptide or protein is prepared. (In some cases, if the nutrient particle size is too large, the nutrient can be passed through a screen (e.g., a SWECO screener)). The nutrient is placed in a volumetric feeder, which delivers a known, accurate concentration of the nutrient (e.g., as a dry powder) at a constant rate.

The liquid fat is added to the slurry vessel at a controlled rate using a metering pump. The rate of addition is selected such that the liquid fat combines with the nutrient in a chosen ratio. For example, if a coated product has 35% of a nutrient and the product is produced at a rate of 100 lbs/hour, the melted fat must be added at a rate of 65 lbs/hour and the volumetric feeder must deliver the nutrient at a rate of 35 lbs/hour.

The melted fat and nutrient are mixed together in the slurry vessel to create an emulsion or suspension. The emulsion/suspension is discharged from the bottom of the vessel and is applied as a layer to a rotating disk underneath the vessel. The emulsion/suspension spreads across the disk because of centrifugal forces. As the layer approaches the edge of the disk, the layer is sheared into discrete particles (i.e., droplets or microcapsules) that contain the nutrient surrounded by the coating. As the particles falls from the disk, the coating cools and solidifies. The coated particle falls into the collection hopper and from the collection hopper onto the transfer conveyor. The conveyor moves the bulk the high melting point coating cools and solidifies. The capsules fall into the collection hopper, down the sides of the collection hopper walls and down onto the transfer conveyor. The conveyor moves the bulk particles to bulk storage for further packaging.

Feed Formulations. Products having an enhanced content of histidine may be included in feed formulation. Tables 3-10 provide examples of feed formulations having an enhanced histidine content.

For example, Table 3 shows one example of a complete feed having an enhanced histidine content. Table 3 lists the relative amounts of feed ingredients that can be used to make up this exemplary complete feed having an enhanced histidine content. The complete feed composition includes a histidine-rich protein which has a histidine content of about 10%. Table 4 lists the amounts of a number of common nutrients that are present in the complete feed composition set forth in Table 3.

Table 5 shows one example of a feed concentrate having an enhanced protein content. Table 5 lists the relative amounts of feed ingredients that can be used to make up this exemplary feed concentrate having an enhanced histidine content. The feed concentrate includes a histidine-rich protein which has a histidine content of about 10%. Table 6 lists the amounts of a number of common nutrients that are present in the feed concentrate set forth in Table 5.

TABLE 3
Complete Feed Having Enhanced Histidine Content, by Ingredient

| Ingredient | Weight Percent |
|---|---|
| Corn, ground fine | 35.75 |
| Wheat midds | 16.54 |
| Soy hulls | 19.95 |
| Soybean Meal, HiPro | 1.88 |
| Salt | 0.5 |
| Molasses | 1.19 |
| Fat | 1.5 |
| Calcium carbonate | 0.715 |
| Cereal Fines | 7.58 |
| Distiller's grains | 10.01 |
| Corn Gluten Meal, 60% | 3.03 |
| Sodium Sesquicarbonate | 0.882 |
| Trace mineral premix | 0.039 |
| Dairy 5 × vitamin premix | 0.031 |
| Magnesium oxide 54 | 0.119 |
| Selenium 0.06% | 0.041 |
| Histidine-rich protein | 0.26 |

TABLE 4
Complete Feed Having High Histidine Content, by Nutrient

| Nutrient | |
|---|---|
| Crude Protein, % | 14.6 |
| Soluble RDP, % | 2.77 |
| RUP, % | 6.25 |
| Fat, % | 4.51 |
| NE_{L}, Mcal/cwt | 79.7 |
| NFC, % | 40.9 |
| ADF, % | 12.4 |
| NDF, % | 22.9 |
| Calcium, % | 0.474 |
| Phosphorus, % | 0.399 |
| Magnesium, % | 0.269 |
| Sulfur, % | 0.185 |
| Salt, % | 0.758 |
| Vitamin A, IU/g | 13.9 |
| Vitamin D, IU/g | 2.12 |
| Vitamin E, IU/kg | 35.4 |
| DDAA HIS, g/kg | 3.06 |
| DDAA LYS, g/kg | 8.02 |
| DDAA MET, g/kg | 2.90 |
| DDAA PHE, g/kg | 5.69 |
| rH | −0.313 |
| Rumen soluble sugar, % | 5.71 |
| Adjusted total starch, % | 29.4 |
| Gelatinized starch, % | 9.09 |
| Digestible NDF, % | 16.6 |

TABLE 5
Feed Concentrate Having Enhanced Protein Content, by
Ingredient

| Ingredient | Weight Percent |
|---|---|
| Rice Bran | 32.00 |
| Ground Corn | 5.00 |
| Soy hulls | 6.125 |
| Feather Meal | 2.00 |
| Soybean Meal, HiPro | 6.367 |
| Salt | 1.701 |
| Calcium Carbonate | 10.437 |
| Magnesium Oxide | 1.54 |
| Corn Gluten Meal, 60% | 22.871 |
| Sodium Bicarbonate | 4.25 |
| Vitamin E | 0.291 |
| Trace Mineral premix | 0.283 |
| Selenium 0.06% | 0.41 |
| Histidine-rich protein | 5.00 |
| Heated soy bean meal | 1.631 |
| Vitamin premix | 0.153 |

TABLE 6
Feed Concentrate Having Enhanced Protein Content, by Nutrient

| Nutrient | |
|---|---|
| Crude Protein, % | 45.55 |
| Soluble RDP, % | 3.18 |
| RUP, % | 28.55 |
| Fat, % | 2.43 |
| NE_{L}, Mcal/cwt | 73.20 |
| NFC, % | 15.21 |
| ADF, % | 3.80 |
| NDF, % | 6.61 |
| Calcium, % | 4.35 |
| Phosphorus, % | 0.36 |
| Magnesium, % | 1.05 |
| Sulfur, % | 0.37 |
| Salt, % | 1.71 |
| Vitamin A, IU/g | 81.4 |
| Vitamin D, IU/g | 10.1 |
| Vitamin E, IU/kg | 225.0 |
| DDAA HIS, g/kg | 9.854 |
| DDAA LYS, g/kg | 12.0 |
| DDAA MET, g/kg | 6.983 |
| DDAA PHE, g/kg | 15.2 |
| Rumen soluble sugar, % | 2.5 |
| Adjusted total starch, % | 9.91 |
| Gelatinized starch, % | 4.1 |
| Digestible NDF, % | 3.5 |

Table 7 shows one example of a supplement having an enhanced content of rumen-protected-histidine. Table 7 lists the relative amounts of feed ingredients that can be used to make up this exemplary supplement. The supplement includes a rumen-protected histidine source, such as rumen protected histidine and/or a rumen protected histidine-rich protein which has a histidine content of about 10%. Table 8 lists the amounts of a number of common nutrients that are present in the supplement set forth in Table 7.

Table 9 shows one example of a complete feed composition having an enhanced content of rumen-protected-histidine. Table 9 lists the relative amounts of feed ingredients that can be used to make up this exemplary feed composition. The feed composition includes a rumen-protected histidine source, such as rumen protected histidine and/or a rumen protected histidine-rich protein which has a histidine content of about 10%. Table 10 lists the amounts of a number of common nutrients that are present in the feed composition set forth in Table 9.

TABLE 7
Supplement With Enhanced Content of Rumen-Protected Histidine,
by Ingredient

| Ingredient | Weight Percent |
|---|---|
| Corn, ground fine | 10.06 |
| Wheat midds | 10.0 |
| Rice Bran | 7.5 |
| Feather Meal | 1.5 |
| Urea | 2.8 |
| Salt | 2.72 |
| Soybean Meal | 0.79 |
| Calcium Carbonate | 6.26 |
| Magnesium Oxide | 1.02 |
| Corn Gluten Meal, 60% | 24.58 |
| Bakery Product | 13.77 |
| Sodium Bicarb | 6.53 |
| Vitamin E | 1.41 |
| Trace mineral premix | .044 |
| Selenium 0.06% | 0.20 |
| Heated Soybean meal | 9.32 |
| Dairy 5X vitamin premix | 0.23 |
| Rumen Protected His | 0.58 |

TABLE 8
Supplement Having Enhanced Content of Rumen-Protected
Histidine, by Nutrient

| Nutrient | |
|---|---|
| Crude Protein, % | 34.0 |
| Soluble RDP, % | 10.63 |
| RUP, % | 14.16 |
| Fat, % | 4.79 |
| NE_{L}, Mcal/cwt | 71.0 |
| NFC, % | 27.12 |
| ADF, % | 3.96 |
| NDF, % | 9.14 |
| Calcium, % | 2.65 |
| Phosphorus, % | 0.44 |
| Magnesium, % | 0.79 |
| Sulfur, % | 0.23 |
| Salt, % | 2.70 |
| Vitamin A, IU/g | 100.21 |
| Vitamin D, IU/g | 15.77 |
| Vitamin E, IU/kg | 775.5 |
| DDAA HIS, g/kg | 5.5 |
| DDAA LYS, g/kg | 6.507 |
| DDAA MET, g/kg | 4.325 |
| DDAA PHE, g/kg | 8.175 |
| Rumen soluble sugar, % | 4.85 |
| Adjusted total starch, % | 19.75 |
| Gelatinized starch, % | 8.56 |
| Digestible NDF, % | 5.69 |

TABLE 9
Complete Feed Having Enhanced Content of Histidine, as
Rumen-Protected Histidine

| Ingredient | Weight Percent |
|---|---|
| Wheat midds | 7.77 |
| Soy hulls | 28.65 |
| Beet Pulp | 11.5 |
| Salt | 0.29 |
| Calcium carbonate | 4.18 |
| Distiller's grains | 13.0 |
| Whole Cotton Seed | 8.0 |
| Wheat flour | 7.70 |
| Canola meal | 5.62 |
| Magnesium oxide 54 | 0.31 |
| Mono-Dicalcium phosphate | 0.58 |
| Corn Gluten Meal, 60% | 0.23 |
| Vitamin E | 0.47 |
| Trace mineral premix | 0.05 |
| Selenium 0.06% | 0.06 |
| Dairy 5x vitamin premix | 0.10 |
| Heat treated soybean meal | 4.5 |
| Rumen bypass histidine | 0.42 |
| Flaked Corn | 10.5 |

TABLE 10
Complete Feed Having Enhanced Histidine Content as Rumen-
Protected Histidine, by Nutrient

| Nutrient | |
|---|---|
| Crude Protein, % | 14.5 |
| Soluble RDP, % | 3.0 |
| RUP, % | 5.93 |
| Fat, % | 4.14 |
| NE_{L}, Mcal/cwt | 69.89 |
| NFC, % | 26.77 |
| ADF, % | 20.47 |
| NDF, % | 21.24 |
| Calcium, % | 2.45 |
| Phosphorus, % | 0.45 |
| Magnesium, % | 0.57 |
| Sulfur, % | 0.68 |
| Salt, % | 0.29 |
| Vitamin A, IU/g | 36.5 |
| Vitamin D, IU/g | 6.67 |
| Vitamin E, IU/kg | 268.5 |
| DDAA HIS, g/kg | 3.98 |
| DDAA LYS, g/kg | 7.11 |
| DDAA MET, g/kg | 2.71 |
| DDAA PHE, g/kg | 4.73 |
| Rumen soluble sugar, % | 5.00 |
| Adjusted total starch, % | 13.00 |
| Gelatinized starch, % | 8.23 |
| Digestible NDF, % | 21.92 |

Construction and Expression of a Histidine-rich protein (HRP) or peptide in a microbial host, Escherichia coli. Construction of a histidine-rich protein construct HrcpET30(Xa/LIC), may be performed as follows. Primers are designed with compatible overhangs for the pET30(Xa/LIC) vector (Novagen, Madison, Wis.) for cloning the Mus musculus histidine-rich calcium binding protein gene (Hrc). The pET vector has a 12 base single stranded overhang on the 5′ side of the Xa/LIC site and a 15-base single stranded overhang on the 3′ side of the Xa/LIC site. The plasmid is designed for ligation independent cloning, with N-terminal His and S-tags and an optional C-terminal His-tag. The Xa protease recognition site (IEGR) sits in front of the start codon of the gene of interest, such that the fusion protein tags can be removed.

The following primers are purchased for pET30 Xa/LIC cloning of the Mus musculus Hrc gene: Forward 5′-GGTATTGAGGGTCGCATGGGCTTCCA GGGGCCATGG-3′ and reverse 5′AGAGGAGAGTTAGAGCCTCACGACCTGTTCTGTTCTC 3′. The nucleic acid sequence of the Mus musculus Hrc gene and corresponding protein sequence are available from GenBank, Accession No. BC021623, as submitted by Strausberg et al., Proc. Natl. Acad. Sci. U.S.A. 99 (26), 16899-16903 (2002), and presented in TABLES 11 and 12. It is possible to design primers that are internal to the Hrc gene such that the peptide that is generated has a higher percentage of histidine residues per total amino acids than the native protein sequence.

TABLE 11

cDNA Sequence of Mus musculus histidine
rich calcium binding protein mRNA

| | |
|---|---|
| 1 | ccacgcgtcc gccaagacct gaggaagata gagaggcaga gagtgggagc tataccacga |
| 61 | caaaagggac aatctgaaag tcaaagccaa aaaggcacaa ggacccatca gaggcagctg |
| 121 | aagccagcct ggtcagacgc tcagctgcta aacgtcccca tgggcttcca ggggccatgg |
| 181 | ttgcacactt gtctcctttg ggccacagtg gccatcctgc tggtccctcc agtggtgacc |
| 241 | caggagttga gaggggccgg tctgggcctg ggcaactgga acaacaatgc aggcatccct |
| 301 | gggtcctcag aggacctatc aactgagttt ggtcaccaca tccaccgggg atatcaaggt |
| 361 | gagaaggaca gaggccacag agaagagggt gaagacttct ccagggaata tggccacagg |
| 421 | gtccaagacc acaggtaccc tggccgcgag gttggagagg agaatgtctc tgaagaggtc |
| 481 | ttcagagggc atgttagaca gctccacggg caccgggaac atgacaatga agatttagga |
| 541 | gactcggcag agaaccacct ccccagacag aggagccaca gccacgaaga tgaggatggc |
| 601 | attgtctcca gtgagtatca ccgtcacgtc cccaggcatg cccaccatgg ccacggagag |
| 661 | gaagatgatg acgatgatgg aggagaggag gaggagaggg tggatgtgat ggaggactct |
| 721 | gatgataatg aacaccaggt ccatggtcac cagagccact caaaggagag agatgaactc |
| 781 | catcatgccc acagccacag gcaccaaggc cacagtgatg atgacgatga cgatggtgtc |
| 841 | tctactgagc atggacacca agctcacaga tatcaggatc atgaggagga agacgatggg |
| 901 | gactcagatg aagacagtca cacocacaga gttcaaggcc gagaagatga aaatgatgat |
| 961 | gaagacggtg actctggtga atacagacac catacccagg accaccaagg ccacaacgaa |
| 1021 | gagcaagatg acgatgatga tgatgatgat gatgatgaag ataaagaaga ctccactgag |
| 1081 | caccggcacc agacccaagg ccacaggaag gaagaagatg aggatgagtc agatgaagat |
| 1141 | gatcatcatg tctccaggca tggacgccaa ggctatgaag aagaagaaga tgatgatgat |
| 1201 | gatgatggag atgatgactc tactgagcat gtgcatcaag cccacagaca cagagaccat |
| 1261 | gagcacaaag atgatgagga tgactcagaa gaagactacc atcatgtccc cggagtcctc |
| 1321 | cggattgctc tctcgactgc cagtggggca gccgctgcct actcagcgcc ttgcctcaac |
| 1381 | ttccccatca gtaccaacac cccctttacc ctcgtgtgga gcctaagaga acagaacagg |
| 1441 | tcgtgaagcc agcaaagaaa agttctgtcg cgtttgtgaa cctttttttt tttttaatca |
| 1501 | aatcgacaac aaacattaaa actttttttt tttaaaaagg acgttaaaaa atttaaaaag |
| 1561 | tatatgagct tcatgggact aactcatcgc cttcccttgc gtacttcaga ttgtagccat |
| 1621 | acttttaaaa aaaaaggcaa agaggataat gacatttttt atcagtattg tgaataaact |
| 1681 | tgaacacaaa tacagaagtt ctatgtcctg tcttcagttg tagaagttgt cttctgcaag |
| 1741 | gtacaaccac ccacttgaac ttcctctgat gacacaatcc acaattctat aagggaatca |
| 1801 | gtgttcacgt ctctgtatat atttatttat gtgtaattta atgggatttg taaatatggt |
| 1861 | gagtctgttt taaacctttt tttatttatc tggtgatctc gtttacctcc tgtttagtgg |
| 1921 | gctttggatc ctccctgtta gttcttcatg tggttttact tagaaatcca aggtttgggt |
| 1981 | aagactcccc ctccccaccc cttttctcca attcatggat ttagccccgt ggtagcatgt |
| 2041 | taaacgatta taatgaaaca gctgaacaaa aacattttta aggtaaaata aaaatttata |
| 2101 | tataattagt aaaaaaaaaa aaaaaaa |

TABLE 12

Amino acid sequence of Mus musculus
histidine rich calcium binding protein

MGFQGPWLHTCLLWATVAILLVPPVVTQELRGAGLGLGNwNNNAGIPGSS
EDLSTEFGHHIHRGYQGEKDRGHREEGEDFSREYGHRVQDHRYPGREVGE
ENVSEEVFRGHVRQLHGHREHDNEDLGDSAENHLPRQRSHSHEDEDGIVS
SEYHRHVPRHAHHGHGEEDDDDDGGEEEERVDVMEDSDDNEHQVHGHQSH
SKERDELHHAHSHRHQGHSDDDDDDGVSTEHGHQAHRYQDHEEEDDGDSD
EDSHTHRVQGREDENDDEDGDSGEYRHHTQDHQGHNEEQDDDDDDDDDDE
DKEDSTEHRHQTQGHRKIEEDEDESDEDDHVSRHGRQGYEEEEDDDDDDG
DDDSTEHVHQAHRHRDHEHKDDEDDSEEDYHHVPGVLRIALSTASGAAAA
YSAPCLNFPISTNTPFTLVWSLREQNRS

It is reported that alterations of tRNA concentrations and aminoacyl-tRNA synthetases reflect the cellular requirements for amino acid biosynthesis. In addition, tRNA can have large effects on the expression and over-expression of heterologous genes in microbial expression systems through reduced translation and errors in amino acid sequences of protein products. (See O'Neill et al., J. Bacteriol. 1990 November; 172(11):6363-7 1); Smith et al., Biotechnol Prog. 1996 July-August; 12(4):417-22); Dieci et al., Protein Expr Purif. 2000 April; 18(3):346-54). Thus, to increase the expression of the histidine-rich proteins for example, it would be beneficial to simultaneously express the corresponding histidyl-tRNA gene as well. It is also possible to design primers to introduce Mus musculus Hrc gene into an operon with HisSpET30 Xa/LIC so that both the histidine-rich calcium binding protein and the histidyl-tRNA synthetase are co-expressed.

Depending on the source of the specific histidine-rich protein, the codon bias of the respective gene could be changed to match the host microbe codon usage in order to achieve higher expression of heterologous proteins. (See Baca et al., Int'l J. of Parasitology. 30: 113-118). Codon usage tables are available from many sources.

Mus musculus histidine-rich calcium binding protein mRNA (cDNA clone MGC: 13723 IMAGE:3979848) is purchased from ATCC, catalog number MGC-13723. All restriction enzymes are purchased from New England BioLabs (Beverly, Mass.). Primers are synthesized by Integrated DNA Technologies, Inc (Coralville, Iowa) unless noted otherwise.

The following is one version of a PCR protocol which can be used to amplify the Mus musculus Hrc gene. In a 50 μL reaction, 0.1-0.5 μg template, 1.5 μM of each primer, 0.4 mM each dNTP, 3.5 U Expand High Fidelity™ Polymerase, and 1×Expand™ buffer with Mg²⁺ were added (Roche, Indianapolis, Ind.). The thermocycler program used includes a hot start at 96° C. for 5 minutes, followed by 29 repetitions of the following steps: 94° C. for 30 seconds, 40-65° C. for 1 minute (gradient thermocycler) and 72° C. for 2 minutes. After the 29 repetitions, the sample is maintained at 72° C. for 10 minutes and then stored at 4° C.

The PCR product is gel purified from 0.8 or 1% TAE-agarose gels using the Qiagen gel extraction kit (Valencia, Calif.). The PCR product is quantified by comparison to standards on an agarose gel, and then treated with T4 DNA polymerase following the manufacturer's recommended protocols for Ligation Independent Cloning (Novagen, Madison, Wis.).

Briefly, about 0.2 pmol of purified PCR product is treated with 1 U T4 DNA polymerase in the presence of dGTP for 30 minutes at 22° C. The polymerase removes successive bases from the 3′ ends of the PCR product. When the polymerase encounters a guanine residue, the 5′ to 3′ polymerase activity of the enzyme counteracts the exonuclease activity to prevent effectively further excision. This creates single stranded overhangs that are compatible with the pET Xa/LIC vector. The polymerase is inactivated by incubating at 75° C. for 20 minutes.

The vector and treated insert are annealed as recommended by Novagen. About 0.02 pmol of treated insert and 0.01 pmol vector are incubated for 5 minutes at 22° C., 6.25 mM EDTA (final concentration) was added, and the incubation at 22° C. is repeated. The annealing reaction (1 μL) was added to NovaBlue™ Singles competent cells (Novagen, Madison, Wis.), and incubated on ice for 5 minutes. After mixing, the cells are transformed by heat shock for 30 seconds at 42° C. The cells are placed on ice for 2 minutes, and allowed to recover in 250 μL of room temperature SOC for 30 minutes at 37° C. with shaking at 225 rpm. Cells are plated on LB plates containing kanamycin (25-50 μg/mL).

Plasmid DNA from cultures that grow on the LB plates with kanamycin is purified using the Qiagen spin miniprep kit (Valencia, Calif.) and screened for the correct inserts. The sequences of plasmids that appeared to have the correct insert are verified by dideoxy chain termination DNA sequencing (SeqWright, Houston, Tex.) with S-tag and T7 terminator primers (Novagen), and internal primers. The sequence verified HrcpET30(Xa/LIC) is transformed into the expression host BL21(DE3) according to Novagen protocols.

Expression of Histidine-rich protein in E. coli BL21(DE3) ::HrcpET30(Xa/LIC) cells may be performed as follows. Fresh plates of E. coli BL21(DE3):: Mus musculus Hrc/pET30(Xa/LIC) cells are prepared on LB medium containing 50 μg/mL kanamycin. Overnight cultures (5 mL) are inoculated from a single colony and grown at 30° C. in LB medium with kanamycin. Typically, a 1 to 5 ml inoculum is used for induction in 100 ml-500 ml LB medium containing 50 μg/mL kanamycin. Cells are grown at 37° C. and sampled every hour until an OD₆₀₀ of 0.35-0.8 is obtained. Cells are then induced with 0.1 mM IPTG. The entire culture volume is centrifuged after approximately 4-10 hours growth (post-induction), for 20 minutes at 4° C. and 3500 rpm. The supernatant is decanted and both the broth and the cells (washed once with sterile distilled water) are separately frozen at −80° C. if immediate analysis is not anticipated. Cell extracts are prepared for protein analysis using Novagen BugBuster™ reagent with benzonase nuclease and Calbiochem protease inhibitor cocktail III according to the Novagen protocol. The level of protein expression in the cell extracts is analyzed by SDS-PAGE using 4-15% gradient gel (Bio-Rad, Hercules, Calif.).

Once the appropriate induction conditions (time, temperature, etc.) that results in maximum histidine-rich protein expression is determined, cells are cultured under those conditions and the cell pellet is resuspended in an appropriate amount of a suitable isotonic buffer, for example, physiological saline (0.85% NaCl pH 7.0). This cell suspension is then lysed using methods known to those skilled in the art, such as French Pressure cells. The lysed cells are centrifuged for 20-3) min at 4oC. at 10,000-15,000 rpm to separate the biomass and cell debris and generate a cell-free extract that contains the Histidine-rich proteins. This extract containing Histidine-rich protein is spray dried to generate a product of Histidine-rich proteins that can be added to animal feed as is or after being subjected to suitable encapsulation to ensure survival through the rumen. Purification and/or concentration of Histidine-rich proteins from E. coli BL21(DE3) ::HrcpET30(Xa/LIC) cells may be performed using techniques described in the literature or detailed below.

Construction and expression of a recombinant or synthetic protein or peptide enriched for histidine (Histidine-rich protein or peptide—HRP) or histidine in combination with selected amino acids combined with expression of appropriate aminoacyl—tRNA synthetase genes. Construction of synthetic a histidine-rich protein or peptide construct HEPpET30(Xa/LIC) may be performed as follows. A synthetic peptide or protein can be designed, for example, to have the following sequence: MHSCNEHPMH LHRPHLHHMH SHHPMGHHSH GHHLHGHHPH SHHLGHHPF GHHPHLHHPH LHHPHGHHPH FHHPHFHDFL DHHHH with content of histidine (H, 44 residues, ˜52%), phenylalanine (F, 4 residues, ˜5%), and leucine (L, 7 residues, ˜8%).

While designing the synthetic gene that will be translated into the desired histidine-rich peptide, the codon usage of the microbial host is taken into consideration so that rare codons are not used. Codon usage in E. coli is expected to be different from that of Corynebacterium for example. An online reference that includes codon reference table is: http://www.kazusa.or.ip/codon/.

Based on a the protocol described in Stemmer et al., Gene 1995 October 16; 164(1):49-53, it is possible to determine the best codons to use to determine the nucleic acid sequence that will encode the desired peptide, design the required number of overlapping oligonucleotides spanning the length of the synthetic nucleic acid, and assemble the synthetic gene using PCR that relies not on DNA ligase but uses the properties of DNA polymerase to build longer DNA fragments during the PCR assembly reaction. The synthetic nucleic acid encoding a histidine-rich peptide can then be cloned into the desired vector containing the appropriate antibiotic/selection marker to ensure expression of the synthetic histidine-rich peptide in the host of choice for example E. coli, Corynebacterium, Brevibacterium, Bacillus, Yeast etc.

It is reported that alterations of tRNA concentrations and aminoacyl-tRNA synthetases reflect the cellular requirements for amino acid biosynthesis, also tRNA can have large effects on the expression and over expression of heterologous genes in microbial expression systems through reduced translation and errors in amino acid sequences of protein products. (See O'Neill et al., J. Bacteriol. 1990 November; 172(11):6363-71; Smith et al., Biotechnol Prog. 1996 July-August; 12(4):417-22); Dieci et al., Protein Expr Purif. 2000 April; 18(3):346-54). Thus, to increase the expression of the synthetic or recombinant histidine-rich proteins, for example, it would be beneficial to simultaneously express the corresponding histidyl-tRNA or respective aminoacyl-tRNA genes as well.

It is also possible to design primers to introduce a synthetic or recombinant gene for Histidine-rich proteins or peptides into an operon with HisSpET30 Xa/LIC so that both the histidine-rich proteins or peptides and the histidyl-tRNA synthetase are co-expressed permitting increased product synthesis.

Expression of synthetic or recombinant Histidine-rich protein or peptide in E. coli BL21(DE3) ::HrcpET30(Xa/LIC) cells may be performed as follows. Fresh plates of E. coli BL21(DE3):: synthetic or recombinant HEP/pET30(Xa/LIC) cells are prepared on LB medium containing 50 μg/mL kanamycin. Overnight cultures (5 mL) are inoculated from a single colony and grown at 30° C. in LB medium with kanamycin. Typically, a 1 to 5 ml inoculum is used for induction in 100 ml-500 ml LB medium containing 50 μg/mL kanamycin. Cells are grown at 37° C. and sampled every hour until an OD₆₀₀ of 0.35-0.8 was obtained. Cells are then induced with 0.1 mM IPTG. The entire culture volume is centrifuged after approximately 4-10 hours growth (post-induction), for 20 minutes at 4° C. and 3500 rpm. The supernatant is decanted and both the broth and the cells (washed once with sterile distilled water) are separately frozen at −80° C. if immediate analysis is not anticipated. Cell extracts are prepared for protein analysis using Novagen BugBuster™ reagent with benzonase nuclease and Calbiochem protease inhibitor cocktail III according to the Novagen protocol. The level of protein expression in the cell extracts is analyzed by SDS-PAGE using 4-15% gradient gel (Bio-Rad, Hercules, Calif.).

Once the appropriate induction time that results in maximum histidine-rich protein or peptide expression is determined, cells are cultured under those conditions and the cell pellet is resuspended in an appropriate amount of a suitable isotonic buffer for example physiological saline (0.85% NaCl pH 7.0). This cell suspension is then lysed using methods known to those skilled in the art, such as French Pressure cells. The lysed cells are centrifuged for 20-30 min at 4° C. at 10,000-15,000 rpm to separate the biomass and cell debris and generate a cell-free extract that contains the Histidine-rich proteins. This extract containing Histidine-rich protein can be spray dried to generate a product of Histidine-rich proteins or peptides that can be added to animal feed as is or after being subjected to suitable encapsulation to ensure survival through the rumen.

Purification or concentration of synthetic or recombinant Histidine-rich proteins from E. coli BL21(DE3) ::HEPpET30(Xa/LIC) cells may be performed if necessary. The histidine-rich proteins or peptides produced can be subjected to further concentration and purification using techniques described in the literature or detailed below.

Construction of Histidine-tRNA synthetase construct HisSpET30(Xa/LIC) may be performed as follows. Primers are designed with compatible overhangs for the pET30(Xa/LIC) vector (Novagen, Madison, Wis.) for cloning the E. coli histidine-tRNA synthetase gene (HisS). The pET vector has a 12 base single stranded overhang on the 5′ side of the Xa/LIC site and a 15-base single stranded overhang on the 3′ side of the Xa/LIC site. The plasmid is designed for ligation independent cloning, with N-terminal His and S-tags and an optional C-terminal His-tag. The Xa protease recognition site (IEGR) sits in front of the start codon of the gene of interest, such that the fusion protein tags can be removed.

The following primers are purchased for pET30 Xa/LIC cloning of the E. coli histidine-tRNA synthetase gene: Forward 5′-GGTATTGAGGGTCGCGtgGCAAAAAACATTCAAGC-3′ and reverse 5′-5′AGAGGAGAGTTAGAGCC TTAACCCAGTAACGTGCGCA-3′. The nucleic acid sequence of the E. coli HisS gene, Accession No. M11843 J01629, is provided in TABLE 13 and the amino acid sequence for the encoded polypeptide is provided in TABLE 14.

TABLE 13
DNA Sequence of E. coli histidine-tRNA synthetase (hisS)

| | |
|---|---|
| 1 | gatatgatcg accagctgga agcacgcatt cgtgcgaaag ccagtcagct ggacgaagcg |
| 61 | cgtcgaattg acgttcagca ggttgaaaaa taataacgtg atgggaagcg cctcgcttcc |
| 121 | cgtgtatgat tgaacccgca tggctcccga aacattgagg gaagcgttga gggttcattt |
| 181 | ttatattcag aaagagaata aacgtggcaa aaaacattca agccattcgc ggcatgaacg |
| 241 | attacctgcc tggcgaaacg gccatctggc agcgcattga aggcacactg aaaaacgtgc |
| 301 | tcggcagcta cggttacagt gaaatccgct tgccgattgt agagcagacc ccgctattca |
| 361 | aacgtgcgat tggtgaagtc accgacgtgg ttgaaaaaga gatgtacacc tttgaggatc |
| 421 | gcaatggcga cagcctgact ctgcgccctg aagggacggc gggctgtgta cgcgccggca |
| 481 | tcgagcatgg tcttctgtac aatcaggaac agcgtctgtg gtatatcggg ccgatgttcc |
| 541 | gtcacgagcg tccgcagaaa gggcgttatc gtcagttcca tcagttgggc tgcgaagttt |
| 601 | tcggtctgca aggtccggat atcgacgctg aactgattat gctcactgcc cgctggtggc |
| 661 | gcgcgctggg tatttccgag cacgtaactc ttgagctgaa ctctatcggt tcgctggaag |
| 721 | cacgcgccaa ttaccgcgat gcgctggtgg cattccttga gcagcataaa gaaaagctgg |
| 781 | acgaagactg caaacgccgc atgtacacta acccgctgcg cgtgctggat tcaaaaaatc |
| 841 | cggaagtgca ggcgcttctc aacgacgctc cggcattagg tgactatctg gacgaggaat |
| 901 | ctcgtgagca ttttgccggt ctgtgcaaac tgctggagag cgcggggatc gcttacaccg |
| 961 | taaaccagcg tctggtgcgt ggtctggatt actacaaccg taccgttttc gagtgggtga |
| 1021 | ctaacagtct cggctcccag ggcaccgtgt gtgcaggcgg tcgttatgac ggtcttgtgg |
| 1081 | aacaactggg cggtcgtgca acaccggctg tcggttttgc tatgggcctc gaacgtcttg |
| 1141 | tattgttagt acaggccgtt aatccggaat ttaaagccga tcctgttgtc gatatatacc |
| 1201 | tggtggcttc aggtgctgat acacaatctg cggctatggc attagctgag cgtctgcgtg |
| 1261 | atgaattacc gggcgtgaaa ttgatgacca accacggcgg cggcaacttt aagaaacagt |
| 1321 | ttgcccgtgc tgataaatgg ggtgcccgcg ttgctgtggt gctgggtgag tctgaagtgg |
| 1381 | ctaacggcac agcagtagtg aaggatttgc gctctggtga gcaaacggca gttgcgcagg |
| 1441 | atagcgtagc cgcgcatttg cgcacgttac tgggttaagg aaggagaagg acagcgtgga |
| 1501 | aatttacgag aacgaaaacg accaggtaga gcggttaaac gcttttttgc tgaaaatggc |
| 1561 | aaagcactgg ctgttggggt gattttggcg ttggcgcact gattggctgg cgctactgga |
| 1621 | acagccatca ggttgattct gcacgctccg cttctcttgc ctatcaaaat gcggttacc |

TABLE 14

Amino Acid Sequence of E. coli
histidine-tRNA synthetase (hisS)

MAKNIQAIRGMNDYLPGETAIWQRIEGTLKNVLGSYGYSEIRLPIVEQTP
LFKRAIGEVTDVVEKEMYTFEDRNGDSLTLRPEGTAGCVRAGIEHGLLYN
QEQRLWYIGPMFRHERPQKGRYRQFHQLGCEVFGLQGPDIDAELIMLTAR
WWRALGISEHVTLELNSIGSLEARANYRDALVAFLEQHKEKLDEDCKRRN
YTNPLRVLDSKNPEVQALLNDAPALGDYLDEESREHFAGLCKLLESAGIA
YTVNQRLVRGLDYYNRTVFEWVTNSLGSQGTVCAGGRYDGLVEQLGGRAT
PAVGFAMGLERLVLLVQAVNPEFKADPVVDIYLVASGADTQSAAMALAER
LRDELPGVKLMTNHGGGNFKKQFARADKWGARVAVVLGESEVANGTAVVK
DLRSGEQTAVAQDSVAAIHLRTLLG

E. coli genomic DNA from Escherichia coli ATCC 10798 is purchased from ATCC, catalog number 10798D. All restriction enzymes are purchased from New England BioLabs (Beverly, Mass.). Primers are synthesized by Integrated DNA Technologies, Inc (Coralville, Iowa) unless noted otherwise.

The following is one version of a PCR protocol is used to amplify the E. coli HisS gene. In a 50 μL reaction, 0.1-0.5 μg template, 1.5 μM of each primer, 0.4 mM each dNTP, 3.5 U Expand High Fidelity™ Polymerase, and IX Expand™ buffer with Mg were added (Roche, Indianapolis, Ind.). The thermocycler program used includes a hot start at 96° C. for 5 minutes, followed by 29 repetitions of the following steps: 94° C. for 30 seconds, 40-65° C. for 1 minute (gradient thermocycler) and 72° C. for 2 minutes, 30 seconds. After the 29 repetitions, the sample is maintained at 72° C. for 10 minutes and then stored at 4° C.

The PCR product is gel purified from 0.8 or 1% TAE-agarose gels using the Qiagen gel extraction kit (Valencia, Calif.). The PCR product is quantified by comparison to standards on an agarose gel, and then treated with T4 DNA polymerase following the manufacturer's recommended protocols for Ligation Independent Cloning (Novagen, Madison, Wis.).

Briefly, about 0.2 pmol of purified PCR product is treated with 1 U T4 DNA polymerase in the presence of dGTP for 30 minutes at 22° C. The polymerase removes successive bases from the 3′ ends of the PCR product. When the polymerase encounters a guanine residue, the 5′ to 3′ polymerase activity of the enzyme counteracts the exonuclease activity to prevent effectively further excision. This creates single stranded overhangs that are compatible with the pET Xa/LIC vector. The polymerase is inactivated by incubating at 75° C. for 20 minutes.

The vector and treated insert are annealed as recommended by Novagen. About 0.02 pmol of treated insert and 0.01 pmol vector are incubated for 5 minutes at 22° C., 6.25 mM EDTA (final concentration) was added, and the incubation at 22° C. is repeated. The annealing reaction (1 μL) is added to NovaBlue™ Singles competent cells (Novagen, Madison, Wis.), and incubated on ice for 5 minutes. After mixing, the cells are transformed by heat shock for 30 seconds at 42° C. The cells are placed on ice for 2 minutes, and allowed to recover in 250 μL of room temperature SOC for 30 minutes at 37° C. with shaking at 225 rpm. Cells are plated on LB plates containing kanamycin (25-50 μg/mL).

Plasmid DNA from cultures that grow on the LB plates with kanamycin is purified using the Qiagen spin miniprep kit (Valencia, Calif.) and screened for the correct inserts. The sequences of plasmids that appeared to have the correct insert are verified by dideoxy chain termination DNA sequencing (SeqWright, Houston, Tex.) with S-tag and T7 terminator primers (Novagen), and internal primers. The sequence verified HisSpET30(Xa/LIC) is transformed into the expression host BL21(DE3) according to Novagen protocols.

Purification of histidine-rich proteins or peptides after a fermentation experiment may be performed as follows. Cells expressing the histidine-rich proteins or peptides are first disrupted using techniques known in the literature for example, using multiple passes through a French press cell at 960 psi on gauge (˜19,000 psi in cell). The cell debris are separated from the HRPs by centrifugation at 15,000 rpm at 4° C. The cell free extract or supernatant contains the HRPs and is subjected to further methods to specifically bind the HRPs and separate them from the other proteins in the cell free extract. One method to purify histidine-rich proteins is based on the ability of a histidine-tag sequence to bind to a histidine binding resin, by binding the histidine-rich protein to the resin and performing metal chelation chromatography principles. A “His Bind Kit” is commercially available from Novagen. The histidine residues and/or histidine-rich segments of the HRPs bind to Ni²⁺ cations which are immobilized on the histidine binding resin. The unbound proteins are washed away and the histidine-rich proteins can be recovered by elution with imidazole. The histidine-rich proteins can be dialyzed to remove the imidazole and then concentrated or spray dried for addition as is, or subjected to appropriate treatment to minimize degradation in the rumen.

All references, patents, and/or applications cited in the specification are indicative of the level of skill of those skilled in the art to which the invention pertains, and are incorporated by reference in their entireties, including any tables and figures, to the same extent as if each reference had been incorporated by reference in its entirety individually.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values are provided for embodiments, additional embodiments are described by taking any 2 different values as the endpoints of a range. Such ranges are also within the scope of the described invention.

1. An animal feed composition comprising an E. coli. biomass derived from fermentation of E. coli. and at least one other nutrient ingredient;
wherein the biomass comprises:
(a) an inhibitor of at least one of the endotoxic effects of lipopolysaccharides of the E. coli.; and
(b) histidine, at least one histidine-rich protein or a mixture thereof.

2. The animal feed composition of claim 1, wherein said biomass has a histidine content of at least 3 mg per gram dry biomass.

3. The animal feed composition of claim 1, wherein the biomass includes at least one histidine-rich protein which is an inhibitor of at least one of the endotoxic effects of lipopolysaccharides of the E. coli.

4. The animal feed composition of claim 1, wherein the at least one histidine-rich protein comprises poly-L-histidine, histidine-rich protein II from Plasmodium falciparum, human histidine-rich glycoprotein, human proline-rich glycoprotein or a mixture thereof.

5. The animal feed composition of claim 1, wherein the at least one histidine-rich protein comprises at least one histidine-rich fragment of histidine-rich protein II from Plasmodium falciparum, at least one histidine-rich fragment of human histidine-rich glycoprotein, at least one histidine-rich fragment of human proline-rich glycoprotein or a mixture thereof.

6. The animal feed composition of claim 1, wherein the other nutrient ingredient includes at least one of fat, fiber, protein and carbohydrate.

7. An E. coli. biomass derived from fermentation of E. coli.; wherein said biomass comprises:
(a) an inhibitor of at least one of the endotoxic effects of lipopolysaccharides of the E. coli.; and
(b) histidine, at least one histidine-rich protein or a mixture thereof.

8. The E. coli biomass of claim 7, wherein said biomass comprises at least one histidine-rich protein which is an inhibitor of at least one of the endotoxic effects of lipopolysaccharides of the E. coli.
